(19) United States
(12) Patent Application Publication
(10) Pub. No.: US 2005/0032182 A1
(43) Pub. Date: Feb. 10, 2005

(54) Process for the enantioselective preparation of secondary alcohols by lipase-catalyzed solvolysis of the corresponding acetoacetic esters

(75) Inventors: Alfred Popp, Untehaching (DE); Jurgen Stohrer, Pullach (DE); Hermann Petersen, Burghausen (DE); Andrea Gilch, Mauern (DE)

Correspondence Address: WILLIAM COLLARD;COLLARD & ROE, P.C., 1077 NORTHERN BOULEVARD, ROSLYN, NY, 11576, US

(73) Assignee: Consortium fur Elektrochemische Industrie GmbH

(21) Appl. No.: 10/912,679

(22) Filed: Aug. 5, 2004

(30) Foreign Application Priority Data
Aug. 7, 2003 (DE) 103 36 270.3

(51) Int. Cl. C12P 7/04; C07D 213/28; C07C 29/94; C07D 207/46

(52) U.S. Cl. 435/157; 546/339; 548/571; 568/702

(57) ABSTRACT

Process for the enantioselective preparation of secondary alcohols, wherein a racemic or enantiomerically enriched mixture of acetoacetic esters of chiral secondary alcohols is subjected to enantioselective enzymatic solvolysis in the presence of a nucleophile and a lipase capable of the solvolytic cleavage of an ester group.

### BACKGROUND OF THE INVENTION

1. Field of the Invention

The invention relates to an enantioselective process for preparing secondary alcohols by enantioselective lipase-catalyzed solvolysis of the corresponding acetoacetic esters.

2. The Prior Art

An ever increasing number of enantiomerically pure compounds serve as starting materials or intermediates in the synthesis of agrochemicals and pharmaceuticals. However, many of these compounds are and were up to now prepared and marketed as racemates or mixtures of diastereomers. In many cases, the desired physiological effect is, however, produced by only one enantiomer or diastereomer. The other isomer is at best inactive, but can also counter the desired effect or even be toxic. For this reason, processes for resolving racemates are becoming increasingly important for the preparation of enantiomerically pure or highly enantiomerically enriched compounds.

It is known that the resolution of racemates of chiral compounds can be carried out with the aid of enzymes. In many publications, the enzymatically kinetic resolution of racemates of esters by means of lipases and esterases is described as a possible method. The resolution of racemates of optically active secondary alcohols is generally carried out by acylation of the hydroxyl group at the stereogenic center or conversely by hydrolysis of the corresponding ester.

The acylation is carried out using, in particular, carboxylic esters for which backreaction of the ester formed is prevented by a subsequent reaction (equilibrium reaction or equilibrium shift by transesterification).

European Patent No. EP 321918 B2 describes the transesterification of a vinyl ester by means of an enzymatically induced enantioselective reaction. In the transesterification, the vinyl alcohols liberated as intermediates then irreversibly form aldehydes or ketones (e.g. acetaldehyde or acetone) which, however, have to be separated in a costly fashion from the reaction mixture since they can inactivate the enzymes used.

This disadvantage is avoided by the use of diketene as an acylating agent to form the corresponding acetoacetic esters, since in this case no cleavage products are formed in the acylation. The use of diketene as an acylating agent is likewise known from the prior-art.

EP 716712 B1 describes a process for preparing enantioselectively acylated alcohols by reacting a racemic alcohol with a diketene in the presence of a specific lipase catalyst. However, only low enantioselectivities are achieved here. Thus, the enzyme-catalyzed reaction of (rac)-1-phenylethanol with diketene results in an enantioselectivity E of only 28, with E being as defined by Chen, C. et al. in J. Am. Chem. Soc. 1982, 104, pp. 7294-7299.

Jeromin, G. et al. in Tetrahedron Lett. 1995, 36(37), pp. 6663-6664 and Suginaka, K. et al. in Tetrahedron: Asymmetry 1996, 7(4), pp. 1153-1158, have also described further processes for preparing enantioselectively acylated alcohols by reaction of a racemic alcohol with a diketene. In these processes, too, only low enantioselectivities (E usually significantly less than 50) are achieved. Furthermore, only low enantiomeric purities and low chemical yields are achieved.

The hydrolysis of acetoacetic esters catalyzed by esterases (Class 3.1.1.1 in accordance with the international enzyme nomenclature, Committee of the International Union of Biochemistry and Molecular Biology) is also known (Lallemand, J.-Y. et al. in Tetrahedron: Asymmetry. 1993, 4(8), pp. 1775-1778). This publication describes the reaction of an acetoacetate with water-catalyzed by pig liver-esterase (PLE) to form the corresponding alcohol. However, the use of esterases brings with it the considerable disadvantage that these (e.g. horse or pig liver esterases) are usually of animal origin and their use is therefore; ruled out in the synthesis of agrochemicals and pharmaceuticals. Furthermore, it has been found (cf. comparative example 3, table 2) that esterases are unsuitable for use in solvolysis reactions, since they give either unsatisfactory selectivities (selectivities E≦2) and/or barely measurable conversions (TOF=0).

A further advantage of the use of diketene as an acylation agent over the transesterification of vinyl esters is that eutomer and distomer can be separated by simple physical methods. The acetoacetic esters formed in the reaction differ significantly in terms of their physical properties (e.g. boiling point, solubility of the Ca²⁺ salts) from the corresponding alcohols and can thus be separated from one another extremely simply (e.g. by distillation, filtration), whereas the vinyl ester and its parent alcohol can be separated by distillation only with great difficulty. The use of acetoacetic esters is therefore preferred especially for the industrial implementation of the resolution of a racemate.

An optimal resolution of a racemate should thus advantageously meet the following conditions:
1. high enantiomeric purity of both enantiomers
2. high selectivity of the enzyme
   (points 1 & 2 reflect a high enantioselectivity E)
3. high chemical yield
4. good space-time yields
5. simple separation of eutomer and distomer
6. ability to be implemented economically on an industrial scale

### SUMMARY OF THE INVENTION

It is an object of the present invention to provide a further enantioselective process for preparing secondary alcohols, which avoids the disadvantages known from the prior art.

This object is achieved by a process in which a nucleophile and a lipase are added to a racemic or enantiomerically enriched mixture comprising enantiomeric acetoacetic esters of chiral secondary alcohols and only one enantiomer in the mixture of enantiomers is solvolyzed. This gives a mixture of an enantiomerically pure or enantiomerically enriched alcohol and the corresponding acetoacetic ester, which can easily be separated from one another.

The invention provides an enantioselective process for preparing secondary alcohols by enantioselective enzymatic solvolysis of a racemic or enantiomerically enriched mixture of the acetoacetic esters of the chiral secondary alcohols in the presence of a nucleophile, which comprises using a lipase capable of the solvolytic cleavage of an ester group.

It has surprisingly been found that significantly higher enantioselectivities can be achieved by enzymatic solvolysis than by the reverse reaction, viz. enzymatic acylation, known from the prior art. Furthermore, damage to the enzyme by undesirable, reactive by-products does not occur and eutomer and distomer can be separated in a simple fashion by simply separating off the appropriate enantiomerically pure or enantiomerically enriched alcohol in the presence of the corresponding acetoacetic ester. The process of the invention thus likewise meets the requirements of an economically attractive process which can readily be implemented on an industrial scale.

The racemic or enantiomerically enriched substrates (acetoacetic esters) used can be prepared very simply from the corresponding alcohols by reaction with diketene (dimerization product of ketene) using methods known from the prior art (cf. Diketenes, Raimund Miller et al. in Ullmann's Encyclopedia of Industrial Chemistry, Vol A15, 5. Ed. (1990), pp. 67-72). The acetoacetic esters can be obtained in excellent purity and yield.

The principle of the process of the invention is illustrated schematically by the general equation (I), in which the radicals R¹ and R² are different so as to generate a chirogenic carbon atom in the parent alcohol and NuH is a general nucleophile:

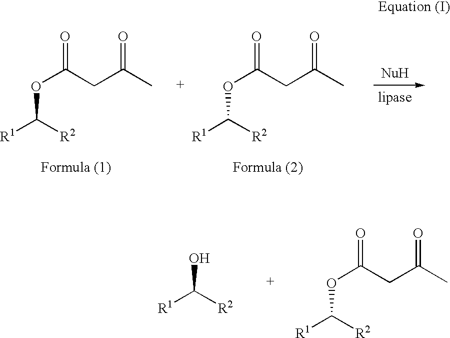

In a preferred embodiment of the invention, a racemic or enantiomerically enriched mixture comprising acetoacetic esters of the general formula (1) and (2), where R¹ is different from R² and the radicals R¹ and R² are selected independently from the group consisting of substituted or unsubstituted C₆-C₁₈-aryls, C₃-C₁₈-heteroaryls, C₁-C₁₈-alkyls, C₂-C₁₈-alkenyls, C₂-C₁₈-alkynyls, C₆-C₁₈-aryl-C₁-C₁₈-alkyls, C₃-C₁₈-heteroaryl-C₁-C₁₈-alkyls, C₆-C₁₈-aryl-C₁-C₁₈-alkenyls, C₃-C₁₈-heteroaryl-C₁-C₁₈-alkenyls, C₁-C₁₈-alkoxy-C₁-C₁₈-alkyls, C₁-C₁₈-alkoxycarbonyls, hydroxycarbonyl, C₁-C₁₈-alkoxy-C₂-C₁₈-alkenyls, C₆-C₁₈-aryloxy-C₁-C₁₈-alkyls, C₆-C₁₈-aryloxy-C₂-C₁₈-alkenyls, C₃-C₈-cycloalkyls, C₃-C₈-cycloalkyl-C₁-C₁₈-alkyls-, C₃-C₈-cycloalkyl-C₂-C₁₈-alkenyls,

or R¹ and R² together with the carbon atom to which they are bound form an asymmetric, substituted, unsubstituted or heteroatom-containing cycloalkylidene and, if the radicals R¹ and R² are substituted radicals, the substituents are selected from the group consisting of unsubstituted or in turn substituted alkyl, alkenyl, alkynyl, aryl, heteroaryl, hydroxy, alkoxy, acyloxy, silyloxy, carboxylate, alkoxycarbonyl, amino, nitro and halogen radicals, is used.

If the abovementioned radicals R¹ and R² contain a heteroatom, this is preferably O, N, S or Si. The radicals R¹ and R² are preferably radicals which form a racemic alcohol having a molecular weight of <300 dalton, since these give eutomer/distomer mixtures which are simple to separate after resolution of the racemate and, for example, the stress of distillation can be avoided in the separation of the alcohol from the remaining acetoacetic ester. Particular preference is given to racemic alcohols in which R¹ and R² differ significantly in their bulk or electrochemical properties, since particularly high selectivities can be achieved in such a case.

Particularly preferred radicals R¹ and R² are selected from the groups consisting of the following radicals: in the group of C₆-C₁₈-aryls, from among: phenyl, naphthyl, methylphenyl, ethylphenyl, propylphenyl, 1-methylethylphenyl, butylphenyl, 1?methylpropylphenyl, 2-methylpropylphenyl, 1,1-dimethylethylphenyl, methylnaphthyl, ethylnaphthyl, propylnaphthyl, 1-methylethylnaphthyl, butylnaphthyl, 1-methylpropylnaphthyl, 2-methylpropylnaphthyl, 1,1-dimethylethylnaphthyl, anthracyl, phenanthryl, o-nitrophenyl, m-nitrophenyl, o-chlorophenyl, m-chlorophenyl, o-bromophenyl, m-bromophenyl, 2,4-dichlorophenyl, 2,3-dichlorophenyl, 3,5-dichlorophenyl, 3,4-dichlorophenyl, 2,4-dimethoxyphenyl, 2,3-dimethoxyphenyl, 3,5-dimethoxyphenyl, 3,4-dimethoxyphenyl, 2-chloro-4-nitrophenyl, o-methoxyphenyl, m-methoxyphenyl, p-chlorophenyl, p-methylphenyl, p-ethylphenyl, p-propylphenyl, p-isopropylphenyl, p-tert-butylphenyl, o-methylphenyl, o-ethylphenyl, o-propylphenyl, o-isopropylphenyl, o-tert-butylphenyl, 4-chloro-2-methylphenyl, p-vinylphenyl, 4-methoxy-2-methylphenyl, o-methoxyphenyl, m-methoxyphenyl and p-methoxyphenyl;

in the group of C₃-C₁₈-heteroaryls, from among: 2-indolyl, 3-indolyl, 2-(4-chloro)thienyl, 2-pyrrolyl, 3-pyrrolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 2-pyridyl, 3-pyridyl, 4-pyridyl, 2-furyl, 3-furyl, 2-thienyl, 3-thienyl, 2-thiazolyl, 4-thiazolyl, 5-thiazolyl, 6-pyridazinyl, C₁-C₁₈-alkyls: methyl, ethyl, n-propyl, 1-methylethyl, n-butyl, 1-methylpropyl, 2-methylpropyl, 1,1-dimethylethyl, n-pentyl, 1—methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, n-nonyl, n-decyl, chloromethyl, dichloromethyl, trichloromethyl, bromomethyl, nitromethyl, hydroxymethyl, chloroethyl, bromoethyl, iodoethyl, aminoethyl, monomethylaminoethyl, dimethylaminoethyl, trimethylsilyloxymethyl, triethylsilyloxymethyl, isopropyldimethylsilyloxymethyl, norbornyldimethylsilyloxymethyl, dinorbornylmethylsilyloxymethyl, triisopropylsilyloxymethyl, tert-butyldimethylsilyloxymethyl, tert-butyldiphenylsilyloxymethyl, 2-chloroethyl, 2-nitroethyl, 2-hydroxyethyl, 2-trimethylsilyloxyethyl, 2-isopropyldimethylsilyloxyethyl, 2-norbornyldimethylsilyloxyethyl, 2-dinorbornylmethylsilyloxyethyl, 2-triisopropylsilyloxyethyl, 2-tert-butyldimethylsilyloxyethyl, 2-tert-butyldiphenylsilyloxyethyl, hydroxycarbonylmethyl, methoxycarbonylmethyl, ethoxycarbonylmethyl, phenyloxycarbonylmethyl, 1-hydroxycarbonyl-1-chloromethyl, 1-methoxycarbonyl-1-methyl, 1-ethoxycarbonyl-1-methyl, acyloxymethyl, acyloxyethyl, ethylcarbonyloxymethyl and propylcarbonyloxymethyl;

in the group of C₂-C₁₈-alkenyls, from among: ethenyl (vinyl), propenyl, 1-butenyl, 2-butenyl, 3-butenyl, 2-methylpropenyl, 1—pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, -2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 2-chlorovinyl, 3-chloro-1-propenyl, 4-chloro-1-butenyl, 5-chloro-1-pentenyl, 6-chloro-1-hexenyl, 7-chloro-1-heptenyl, 8-chloro-1-octenyl, 9-chloro-1-nonenyl, 10-chloro-1-decenyl, 2-nitrovinyl, 3-nitro-1-propenyl, 4-nitro-1-butenyl, 5-nitro-1-pentenyl, 6-nitro-1-hexenyl, 7-nitro-1-heptenyl, 8-nitro-1-octenyl, 9-nitro-1-nonenyl, 2-propenyl, 2-butenyl, 1-methyl-2-propenyl, 2-methyl-2-propenyl, 10-nitro-1-decenyl, 1,2-dimethyl-2-hexenyl, 1-ethyl-2-butenyl;

in the group of C₂-C₁₈-alkynyls: ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methyl-but-1-yn-3-yl, 3-methyl-but-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl, 4-methylpent-2-yn-5-yl, 3-chloro-1-propynyl, 4-chloro-1-butynyl, 5-chloro-1-pentynyl, 6-chloro-1-hexynyl, 7-chloro-1-heptynyl, 8-chloro-1-octynyl, 9-chloro-1-nonynyl, 10-chloro-1-decynyl, 2-nitrovinyl, 3-nitro-1-propynyl, 4-nitro-1-butynyl, 5-nitro-1-pentynyl, 6-nitro-1-hexynyl, 7-nitro-1-heptynyl, 8-nitro-1-octynyl, 9-nitro-1-nonynyl, 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 10-nitro-1-decynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl and 1-ethyl-1-methyl-2-propynyl;

in the group of C₆-C₁₈-aryl-C₁-C₁₈-alkyls, from among: benzyl, naphthylmethyl, o-nitrophenylmethyl, m-nitrophenylmethyl, o-chlorophenylmethyl, m-chlorophenylmethyl, 2,4-dichlorophenylmethyl, 2,3-dichlorophenylmethyl, 3,5-dichlorophenylmethyl, 3,4-dichlorophenylmethyl, 2,4-dimethoxyphenylmethyl, 2,3-dimethoxyphenylmethyl, 3,5-dimethoxyphenylmethyl, 3,4-dimethoxyphenylmethyl, 2-chloro-4-nitrophenylmethyl, o-methoxyphenylmethyl, m-methoxyphenylmethyl, p-chlorophenylmethyl, p-methylphenylmethyl, p-ethylphenylmethyl, p-propylphenylmethyl, p-isopropylphenylmethyl, 4-chloro-2-methylphenylmethyl, p-vinylphenylmethyl, phenylethyl, naphthylethyl, o-nitrophenylethyl, m-nitrophenylethyl, o-chlorophenylethyl, m-chlorophenylethyl, 2,4-dichlorophenylethyl, 2,3-dichlorophenylethyl, 3,5-dichlorophenylethyl, 3,4-dichlorophenylethyl, o-methoxyphenylethyl, m-methoxyphenylethyl, p-chlorophenylethyl, p-methylphenylethyl, p-ethylphenylethyl, p-propylphenylethyl and p-vinylphenylethyl;

in the group of C₃-C₁₈-heteroaryl-C₁-C₁₈-alkyls, from among: 2-indolylmethyl, 3-indolylmethyl, 2-pyrrolmethyl, 3-pyrrolmethyl, 2-pyridylmethyl, 3-pyridylmethyl, 2-furylmethyl, 3-furylmethyl, 2-imidazolylmethyl, 4-imidazolylmethyl, 5-imidazolylmethyl, 3-pyridazinylmethyl, 4-pyridazinylmethyl and 5-pyridazinylmethyl;

in the group of C₆-C₁-aryl-C₁-C₁₈-alkenyls, from among: styryl, 2-phenylpropenyl, 1-phenyl-1-butenyl, 2-phenyl-1-butenyl, 3-phenyl-1-butenyl, 1-phenyl-2-butenyl, 2-phenyl-2-butenyl, 3-phenyl-2-butenyl, 1-phenyl-3-butenyl, 2-phenyl-3-butenyl, 3-phenyl-3-butenyl, 1-phenyl-1-pentenyl, 2-phenyl-1-pentenyl, 3-phenyl-1-pentenyl, 4-phenyl-1-pentenyl, 1-phenyl-2-pentenyl, 2-phenyl-2-pentenyl, 3-phenyl-2-pentenyl, 4-phenyl-2-pentenyl, 1-phenyl-3-pentenyl, 2-phenyl-3-pentenyl, 3-phenyl-3-pentenyl, 4-phenyl-3-pentenyl, 1-phenyl-4-pentenyl, 2-phenyl-4-pentenyl, 3-phenyl-4-pentenyl, 4-phenyl-4-pentenyl, 1-nitrophenyl-1-butenyl, 2-nitrophenyl-1-butenyl, 3-nitrophenyl-1-butenyl, 1-nitrophenyl-2-butenyl, 2-nitrophenyl-2-butenyl, 3-nitrophenyl-2-butenyl, 1-nitrophenyl-3-butenyl, 2-nitrophenyl-3-butenyl, 3-nitrophenyl-3-butenyl, 1-methoxyphenyl-1-butenyl, 2-methoxyphenyl-1-butenyl, 3-methoxyphenyl-1-butenyl, 1-methoxyphenyl-2-butenyl, 2-methoxyphenyl-2-butenyl, 3-methoxyphenyl-2-butenyl, 1-methoxyphenyl-3-butenyl, 2-methoxyphenyl-3-butenyl, 3-methoxyphenyl-3-butenyl, 1-chlorophenyl-1-butenyl, 2-chlorophenyl-1-butenyl, 3-chlorophenyl-1-butenyl, 1-chlorophenyl-2-butenyl, 2-chlorophenyl-2-butenyl, 3-chlorophenyl-2-butenyl, 1-chlorophenyl-3-butenyl, 2-chlorophenyl-3-butenyl and 3-chlorophenyl-3-butenyl;

in the group of C₃-C₁₈-heteroaryl-C₁-C₁₈-alkenyls, from among: 2-indolylpropenyl, 1-indolyl-1-butenyl, 2-indolyl-1-butenyl, 3-indolyl-1-butenyl, 1-indolyl-2-butenyl, 2-indolyl-2-butenyl, 3-indolyl-2-butenyl, 1-indolyl-3-butenyl, 2-indolyl-3-butenyl, 3-indolyl-3-butenyl, 2-pyrrolylpropenyl, 1-pyrrolyl-1-butenyl, 2-pyrrolyl-1-butenyl, 3-pyrrolyl-1-butenyl, 1-pyrrolyl-2-butenyl, 2-pyrrolyl-2-butenyl, 3-pyrrolyl-2-butenyl, 1-pyrrolyl-3-butenyl, 2-pyrrolyl-3-butenyl, 3-pyrrolyl-3-butenyl, 2-pyrimidylpropenyl, 1-pyrimidyl-1-butenyl, 2-pyrimidyl-1-butenyl, 3-pyrimidyl-1-butenyl, 1-pyrimidyl-2-butenyl, 2-pyrimidyl-2-butenyl, 3-pyrimidyl-2-butenyl, 1-pyrimidyl-3-butenyl, 2-pyrimidyl-3-butenyl, 3-pyrimidyl-3-butenyl, 2-imidazolylpropenyl, 1-imidazolyl-1-butenyl, 2-imidazolyl-1-butenyl, 3-imidazolyl-1-butenyl, 1-imidazolyl-2-butenyl, 2-imidazolyl-2-butenyl, 3-imidazolyl-2-butenyl, 1-imidazolyl-3-butenyl, 2-imidazolyl-3-butenyl, 3-imidazolyl-3-butenyl, 2-pyridylpropenyl, 1-pyridyl-1-butenyl, 2-pyridyl-1-butenyl, 3-pyridyl-1-butenyl, 1-pyridyl-2-butenyl, 2-pyridyl-2-butenyl, 3-pyridyl-2-butenyl, 1-pyridyl-3-butenyl, 2-pyridyl-3-butenyl and3-pyridyl-3-butenyl;

in the group of C₁-C₁₈-alkoxy-C₁-C₁₈-alkyls, from among: methoxymethyl, ethoxymethyl, methoxyethyl, ethoxyethyl and benzyloxymethyl;

in the group of C₁-C₁₈-alkoxy-C₂-C₁₈-alkenyls, from among: methoxybutenyl, ethoxybutenyl and benzyloxybutenyl;

in the group of C₆-C₁₈-aryloxy-C₁-C₁₈-alkyls: phenyloxymethyl;

in the group of C₆-C₁₁-aryloxy-C₂-C₁₈-alkenyls: phenyloxybutenyl;

in the group of C₃-C₈-cycloalkyls, from among: cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentylcyclopropyl, 1-methyl-1-butylcyclopropyl, 1,2-dimethylcyclyopropyl, 1-methyl-2-ethylcyclopropyl, cyclooctyl, cyclononyl, cyclodecyl, cyclopropenyl, cyclobutenyl, cyclopentenyl, cyclohexenyl, cycloheptenyl and cyclooctenyl;

in the group of C₃-C₈-cycloalkyl-C₁-C₁₈-alkyls, from among: cyclopropylmethyl, cyclobutylmethyl, cyclopentylmethyl, cyclohexylmethyl;

in the group of C₃-C₈-cycloalkyl-C₂-C₁₈-alkenyls: cyclopropyl-1-butenyl, cyclobutyl-1-butenyl, cyclopentyl-1-butenyl, cyclobutenyl-1-butenyl and cyclopentenyl-1-butenyl;

in the group of C₁-C₁₈-alkoxycarbonyls: methoxycarbonyl, ethoxycarbonyl and benzyloxycarbonyl;

in the group of cycloalkylidenes, from among: 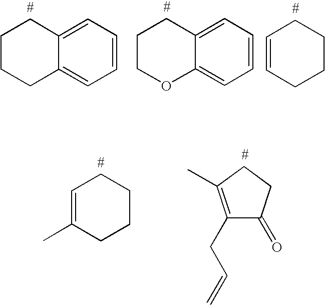
where # in each case marks the point of linkage of the alkylidene.

Particularly preferred substrates are the acetoacetic esters of the following racemic alcohols: 2-butanol, 2-pentanol, 2-hexanol, 2-heptanol, 2-octanol, 1-phenylethanol, 1-phenyl-1-propanol, 1-phenyl-2-propanol, 1-(4-chlorophenyl)ethanol, 1-(4-chlorophenyl)propanol, 2-chloro-1-phenylethanol, 3-chloro-1-phenylpropanol, 2-chloro-1-(4-chlorophenyl)ethanol, 3-chloro-1-(4-chlorophenyl)propanol, 2-chloro-1-(3-chlorophenyl)ethanol, 3-chloro-1-(3-chlorophenyl)propanol, 2-chloro-1-(2-chlorophenyl)ethanol, 3-chloro-1-(2-chlorophenyl)propanol, 1-(4-nitrophenyl)ethanol, 1-(4-nitrophenyl)propanol, 1-naphthylethanol, 1-naphthylpropanol, 1-(6-methoxynaphthyl)ethanol, 1-(6-methoxynaphthyl)propanol, 2-chloro-1-naphthylethanol, 3-chloro-1-naphthylpropanol, 2-chloro-1-(6-methoxynaphthyl)ethanol, 3-chloro-1-(6-methoxynaphthyl)propanol, 1-(4-methylphenyl)ethanol, 1-(4-methylphenyl)propanol, 2-chloro-1-(4-methylphenyl)ethanol, 3-chloro-1-(4-methylphenyl)propanol, 1-(4-ethylphenyl)ethanol, 1-(4-ethylphenyl)propanol, 2-chloro-1-(4-ethylphenyl)ethanol, 3-chloro-1-(4-ethylphenyl)propanol, 1-(4-methoxyphenyl)ethanol, 1-(4-methoxyphenyl)propanol, 2-chloro-1-(4-methoxyphenyl)ethanol, 3-chloro-1-(4-methoxyphenyl)propanol, 1-(2-methylphenyl)ethanol, 1-(2-methylphenyl)propanol, 2-chloro-1-(2-methylphenyl)ethanol, 3-chloro-1-(2-methylphenyl)propanol, 1-(2-ethylphenyl)ethanol, 1-(2-ethylphenyl)propanol, 2-chloro-1-(2-ethylphenyl)ethanol, 3-chloro-1-(2-ethylphenyl)propanol, 1-(2-methoxyphenyl)ethanol, 1-(2-methoxyphenyl)propanol, 2-chloro-1-(2-methoxyphenyl)ethanol, 3-chloro-1-(2-methoxyphenyl)propanol, 1-(3-methylphenyl)ethanol, 1-(3-methylphenyl)propanol, 2-chloro-1-(3-methylphenyl)ethanol, 3-chloro-1-(3-methylphenyl)propanol, 1-(3-ethylphenyl)ethanol, 1-(3-ethylphenyl)propanol, 2-chloro-1-(3-ethylphenyl)ethanol, 3-chloro-1-(3-ethylphenyl)propanol, 1-(3-methoxyphenyl)ethanol, 1-(3-methoxyphenyl)propanol, 2-chloro-1-(3-methoxyphenyl)ethanol, 3-chIoro—1-(3-methoxyphenyl)propanol, 1-(1,3)-benzodioxolethanol, 5-phenylpent-1-en-3-ol, 4-phenylbut-3-en-2-ol, 1-(6-methoxy-2-naphthyl)ethanol, 1-(4-isobutylphenyl)ethanol, 1-pyridin-3-ylethanol, 1-(2-furyl)ethanol, 2-bromo-1-(4-nitrophenyl)ethanol, cyclopropyl(phenyl)methanol, 3-chloro-1-thien-2-ylpropan-1-ol, 3-iodo-1-thien-2-ylpropan-1-ol, 3-(methylamino)-1-thien-2-ylpropan-1-ol, 3-(dimethylamino)-1-thien-2-ylpropan-1-ol, methyl 3-ethyl-3-(hydroxymethyl)heptanoate, 1-trimethylsilanyloxypropan-2-ol, 1-triisopropylsilanyloxypropan-2-ol, 1-(tert-butyldimethylsilanyloxy)propan-2-ol, 1-(tert-butyldiphenylsilanyloxy)propan-2-ol, 1-(tert-butyldiphenylsilanyloxy)butan-2-ol, 1-(tert-butyldiphenylsilanyloxy)-3-chloropropan-2-ol, 1-(2-norbornyldimethylsilanyloxy)propan-2-ol, 1-(2-norbornyldimethylsilanyloxy)-3-chloropropan-2-ol, 1,2,3,4-tetrahydronaphthalen-1-ol, chroman-4-ol, cyclohex-2-enol, 3-methylcyclohex-2-enol, 2-allyl-4-hydroxy-3-methylcyclopent-2-enone, methyl 3-hydroxybutanoate, ethyl 3-hydroxybutanoate, methyl 4-chloro-3-hydroxybutanoate and ethyl 4-chloro-3-hydroxybutanoate.

The acetoacetic esters of secondary alcohols which are to be used as starting materials in the process of the invention are preferably obtained by reacting racemic or enantiomerically enriched mixtures of the secondary alcohols with diketene using the methods known from the prior art.

In general, nucleophiles which can be used in the process of the invention have-the-general formula NuH, where Nu can be OR⁵, SR⁵, or NR⁶R⁷ and
R⁵ is selected from the group consisting of hydrogen, substituted or unsubstituted C₁-C₁₈-alkyl, C₂-C₁₈-alkenyl, C₂-C₁₈-alkynyl, C₆-C₁₈-aryl-C₁-C₁₈-alkyl, C₃-C₁₈-heteroaryl-C₁-C₁₈-alkyl, C₆-C₁₈-aryl-C₂-C₁₈-alkenyl, C₃-C₁₈-heteroaryl-C₂-C₁₈-alkenyl and
R⁶ and R⁷ may be identical or different and are selected independently from the group consisting of hydrogen, substituted or unsubstituted C₁-C₁₈-alkyl, C₂-C₁₈-alkenyl, C₂-C₁₈-alkynyl C₆-C₁₈-aryl, C₃-C₁₈-heteroaryl, C₆-C₁₈-aryl-C₁-C₁₈-alkyl, C₃-C₁₈-heteroaryl-C₁-C₁₈-alkyl, C₆-C₁₈-aryl-C₂-C₁₈-alkenyl, C₃-C₁₈-heteroaryl-C₂-Cle-alkenyl, and
if the radicals R⁵, R⁶ and R⁷ are substituted radicals, the substituents are selected from the group consisting of unsubstituted or in turn substituted alkyl, alkenyl, alkynyl, aryl, heteroaryl, hydroxy, alkoxy, acyloxy, silyloxy, carboxylate, alkoxycarbonyl, amino, nitro and halogen radicals.

If the abovementioned radicals R⁵, R⁶ and R⁷ contain a heteroatom, this is preferably O, N, S or Si.

In a preferred embodiment of the process of the invention, the nucleophile NuH is an oxygen-containing compound HOR⁵.

The nucleophile is particularly preferably a branched or unbranched C₁-C₆-alcohol or water (R⁵=H), in particular methanol, ethanol, n-propanol, isopropanol or butanol.

All lipases which are capable of cleaving an ester bond are in principle suitable for use in the process of the invention. The lipase is preferably a lipase of class 3.1.1.3 in accordance with the international enzyme nomenclature, Committee of the International Union of Biochemistry and Molecular Biology. Due to their relatively ready availability, particular preference is given to lipases of microbial origin. Lipases of microbial origin which may be mentioned by way of example are lipases from fungi, yeasts or bacteria, for example from Alcaligenes sp. (BTL2), Aspergillus niger (ANL), Aspergillus oryzae, Bacillus sp, Bacillus stearothermophilus, Bacillus thermoglucosidasius, Candida antarctica (CAL), Candida lipolytica (CLL), Candida rugosa (CRL), Chromobacterium viscosum (CVL), Geotrichum scandium (GCL), Mucor miehei (MML), Penicillium camembertii (PcamL), Penicillium roquefortii (ProqL), Pseudomonas cepacia (PCL), Pseudomonas fluorescens (PFL), Pseudomonas sp. (PSL), Rhizomucor javanicus (RJL), Rhizopus arrhizus (RAL), Rhizopus niveus (RNL), Saccharomyces cerevisiae, Thermoanaerobium brockii, Thermomyces lanuginosa (TLL).

Particular preference is given to lipases from. Candida species: such as Candida antarctica B (CAL-B). Very particularly preferred enzymes are Novozym® 435, 525 (Novo, Denmark) and Chirazyme® L2 (Böhringer Mannheim, Germany).

The enzymes are used in the reaction either directly or in immobilized form (as immobilisates) on a variety of insoluble supports, in adsorptively or covalently bound form. The immobilisates can be prepared by dissolution of the enzyme in a buffer at a suitable pH and subsequent passive adsorption on the supports, e.g. diatomaceous earth (Celite®), activated carbon, aluminum oxide, silica gel, kieselguhr, monodisperse soluble organosiloxane particles or resins (e.g. Amberlite®, Dowex®). As an alternative, the enzymes can also be covalently bound to the support (e.g. polystyrene or epoxy resins such as Eupergit®). The supported enzymes can be dried by lyophilization.

The amount of lipase to be used in the process of the invention generally depends on the type of starting material, the type of product and the activity of the enzyme preparation. The amount of enzyme which is optimal for the reaction can easily be determined by a person skilled in the art by means of simple preliminary tests.

Depending on the lipase, the enzyme/substrate ratio-calculated as the molar ratio of enzyme to substrate is generally, from 1:1000 to 1:50,000,000, preferably from 1:10,000 to 1:5,000,000.

The enantioselectivity E of the lipases is generally in the range from 5 to >100. The enantioselectivity is preferably greater than 10.

In the process of the invention, the solvent can function directly as nucleophile (NuH). As an alternative, it is also possible to use mixtures of the nucleophile (NuH) with one or more aprotic or protogenic solvents, as long as the solvent or solvent mixtures do not affect the reactivity of the enzyme or lead to undesirable secondary reactions. The reaction is advantageously carried out in a mixture of the nucleophile (NuH) and a suitable inert solvent.

The inert solvent is preferably selected from the group consisting of aliphatic hydrocarbons, aromatic hydrocarbons, halogenated hydrocarbons, ethers, alcohols which are not nucleophiles for the purposes of the abovementioned enzymatic reaction, esters and acetonitrile and mixtures of the solvents mentioned.

From the group of hydrocarbons, particular preference is given to hexane, cyclohexane, petroleum ether or toluene. From the group of halogenated hydrocarbons, preference is given to methylene chloride or chloroform. From the group of ethers, preference is given to methyl tert-butyl ether. (MTBE), THF, diethyl ether, diisopropyl ether or dioxane. Alcohols which are not nucleophiles for the purposes of the abovementioned enzymatic reaction are, in particular, tertiary alcohols such as tert-butanol.

A particularly preferred reaction medium is a mixture of water, n-propanol or isopropanol (as nucleophile NuH) and an inert solvent, especially the abovementioned preferred solvents, in particular MTBE. The ratio of nucleophile to solvent (v/v) is preferably in a range from 1:10,000 to 10,000:1.

Particular preference is given to mixtures of the nucleophile with aprotic solvents such as methyl tert-butyl ether (MTBE) or diisopropyl ether in a ratio of nucleophile/solvent (v/v) of from 1:100 to 100:1.

If water is used as nucleophile (Nu=OH), a prescribed pH can be obtained by adjusting it and keeping it constant by addition of a buffer. Preference is given to a Na₂HPO₄/NaH₂PO₄ buffer having a pH of 7.0. An aqueous alkali, preferably a solution of an alkali metal hydroxide in water, particularly preferably an aqueous solution of NaOH or KOH, can also be added for the same purpose.

The reaction is advantageously carried out at a temperature of from 0° C. to 75° C., preferably from 10° C. to 60° C., particularly preferably from 20° C. to 50° C.

Depending on the substrate, ester and type and amount of enzyme, the reaction times are from 10 minutes to 7 days. The reaction times are preferably in the range from 1 to 48 hours.

The course of the reaction can readily be followed by customary methods, for example by means of GC, HPLC or the consumption of alkali in the constant pH titration. The reaction can be stopped according to a desired result (high conversion, high enantiomeric excess of the substrate or of the product). In the ideal case, the reaction is stopped at a conversion of 50% at a high enantiomeric purity both in the substrate and in the product.

The reaction is advantageously stopped by, for example, separation of the substrate or product from the enzyme, e.g. by extraction, filtration or distillation. The reaction can also be terminated by deactivation of the enzyme, e.g. by thermal or chemical degradation.

If the reaction is carried out by repeated, continuous pumping of the reaction solution through a container filled with enzyme, the reaction is preferably stopped by stopping the pumped circulation.

Depending on the enzyme, the (R) or (S) stereoisomer (cf. general equation (I)) of the ester is solvolyzed and the corresponding free alcohol is formed, selectively. The other enantiomer in each case is not reacted and remains unchanged in the form of its acetoacetic ester.

The product mixture obtained can easily be separated by distillation into eutomer and distomer because of the significant boiling point differences (cf. table (1)) between ester and alcohol. This makes it possible to achieve quantitative separation of the products, which is in turn a prerequisite for achieving optical purity. Thus, in a preferred embodiment, the lower-boiling alcohol can be distilled off, leaving the acetoacetic ester in the bottoms. The acetoacetic ester can subsequently be purified in a separate distillation or after hydrolysis.

In equation (II) (cf. example 1), the process is outlined by way of example for the conversion according to the invention of a racemic mixture of the acetoacetic ester of 1-phenylethanol into the (R) enantiomer of 1-phenylethanol (Ia) to give the corresponding ester of (S)-1-phenylethanol (IIb) having the opposite chirality and its further conversion into (S)-1-phenylethanol (Ib) in a subsequent step comprising separation of enantiomers and hydrolysis.

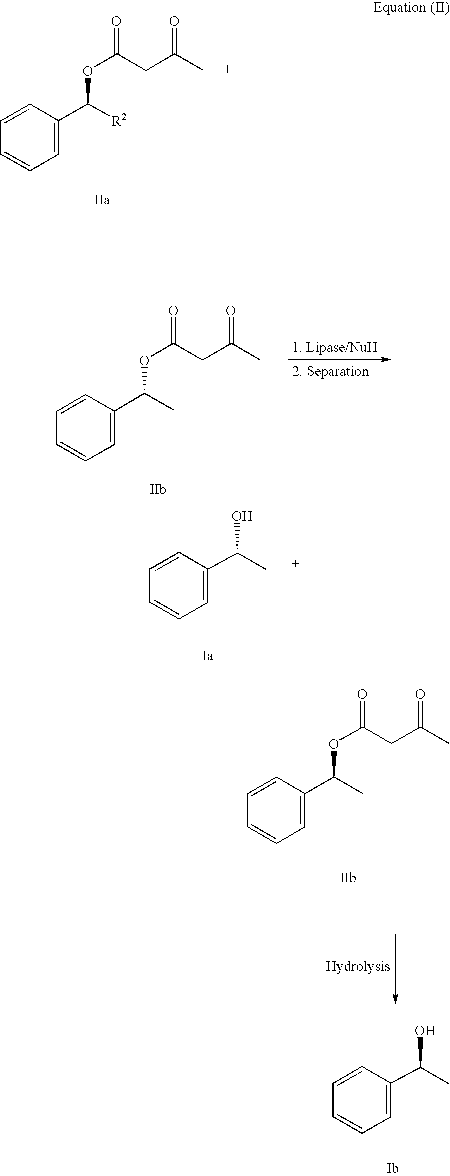

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows the course (conversion versus time) of the CAL-B-catalyzed conversion of: (rac)-1-phenylethyl. 3-oxobutanoate into (1R)-1-phenylethanol and (1S)-1-phenylethyl 3-oxobutanoate; and

FIG. 2 shows the selectivity E of the CAL-B-catalyzed conversion of (rac)-1-phenylethyl 3-oxobutanoate into (1R)-1-phenylethanol and (1S)-1-phenylethyl 3-oxobutanoate and the enantiomeric excess (ee) as a function of the conversion (c).

### DETAILED DESCRIPTION OF THE PREFERRED EMBODIMENT

The invention will now be described with reference to the following examples:

### EXAMPLE 1

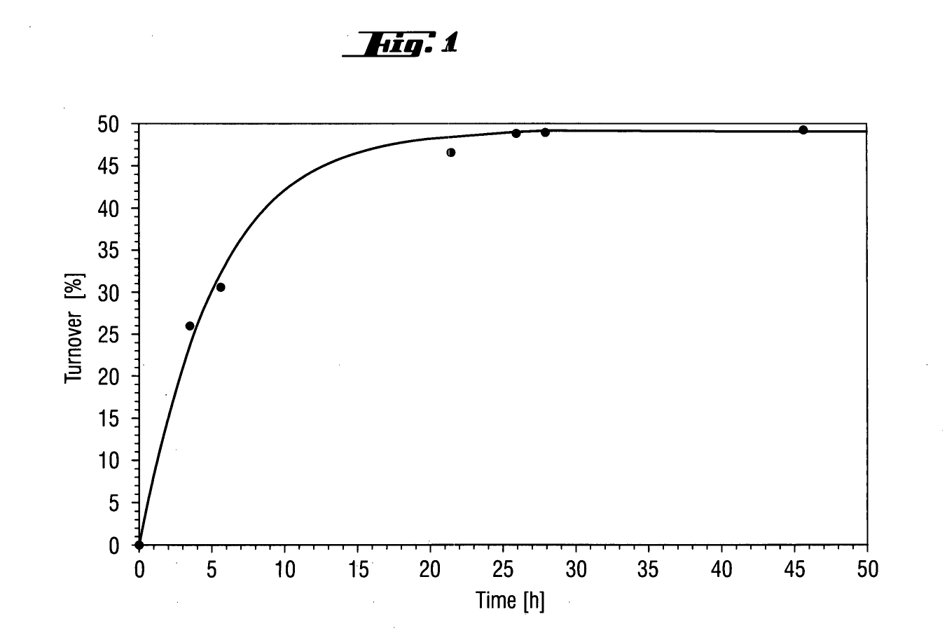

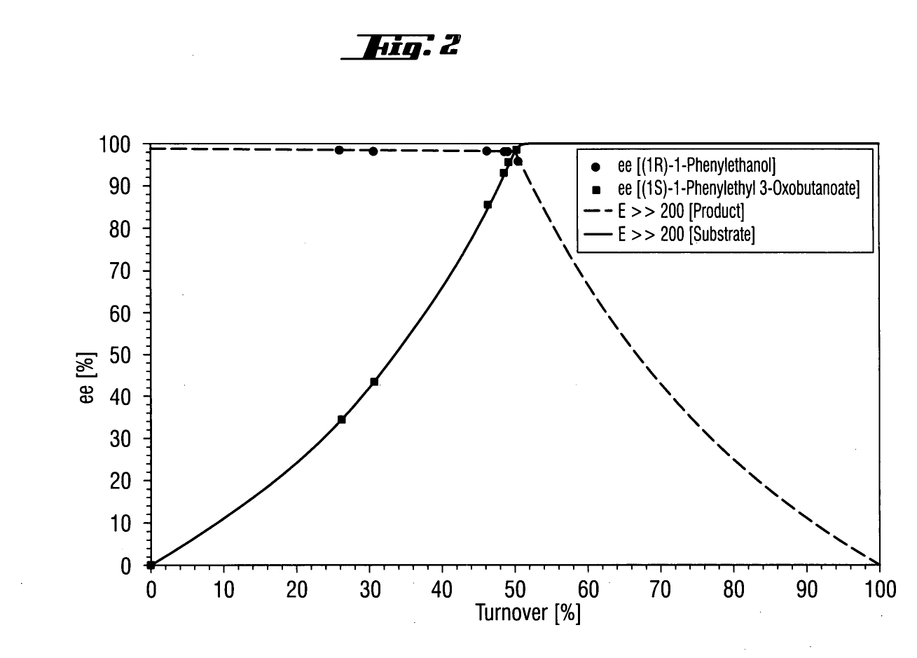

A solution of (rac)-1-phenylethyl 3-oxobutanoate (16% (w/w)) in a mixture of isopropanol and MTBE (1:1 (v/v)) is thermostated to 40° C. The reaction is started by addition of Novozym® 435 (3% (w/w) based on the racemate). Samples are taken at regular intervals and the ee of substrate and product is measured or the conversion is determined (cf. FIGS. 1 and 2). After a conversion of about 50% has been reached, the reaction is interrupted by filtering off the enzyme. The organic phase is then evaporated under reduced pressure. The residue comprises (1R)-1-phenylethanol (ee=98%) and (1S)-1-phenylethyl 3-oxobutanoate (ee=96%). The two compounds are separated from one another by distillation. (1S)-1-Phenylethyl. 3-oxobutanoate is subsequently hydrolyzed by known methods to give (1S)-1-phenylethanol as product.

### EXAMPLE 2

All the following examples (table (1): 1a-h, 2a-e, 3a-1, 4a-1, 5a-f) serve to illustrate the present invention further and were carried out according to the following general method:

A solution of the racemic acetoacetic ester (about 16% (w/w)) in a mixture of the appropriate nucleophile (see Nu in table (1)) and, if desired, a cosolvent (cf. table (1)) is thermostated to 40° C. The reaction is started by addition of the appropriate enzyme. The reaction is stopped by filtering off the enzyme. The organic phase is then evaporated under reduced pressure. The two compounds are separated from one another by distillation.

TABLE (1)

| | Substituents | | | | | Selectivity E | | | | |
|---|---|---|---|---|---|---|---|---|---|---|
| | corresponding | | | | | by the | TOF | | | |
| | to | | En- | | Cosolvent | method | [mmol | B.p. of | B.p. of | B.p. |
| | formula (I) | Racemic substrate | zyme | Nu | [% (v/v)] | of Sih¹ | g⁻¹h⁻¹] | substrate | product | difference |
| 1a | R¹ = C₆H₅R² = CH₃ | 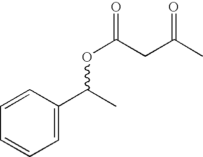 | CAL- B | OH | — | >100 | 32 | 150° C. @ 13 mbar | 85° C. @ 13 mbar | 65° C. |
| 1b | | | PCL | OH | — | >100 | 1 | | | |
| 1c | | | PFL | OH | — | >100 | 145 | | | |
| 1d | | | CAL- | OCH₃ | MTBE | >100 | 2 | | | |
| | | | B | | [50] | | | | | |
| 1e | | | CAL- | OCH₂CH₃ | MTBE | >100 | 4 | | | |
| | | | B | | [50] | | | | | |
| 1f | | | CAL- | O(CH₂)₂CH₃ | MTBE | >100 | 5 | | | |
| | | | B | | [50] | | | | | |
| 1g | | | CAL- | OCH(CH₃)₂ | MTBE | >100 | 7 | | | |
| | | | B | | [50] | | | | | |
| 1h | | | CAL- | O(CH₂)₃CH₃ | MTBE | >100 | 5 | | | |
| | | | B | | [50] | | | | | |
| 2a | R¹ =(CH₂)₂CH₃R² = CH₃ | 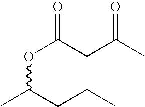 | CAL- B | OH | — | 30 | 23 | 212° C. @ 1013 mbar | 120° C. @760 mbar | 92° C. |
| 2b | | | CAL- | O(CH₂)₂CH₃ | — | 40 | 4 | | | |
| | | | B | | | | | | | |
| 2c | | | CAL- | O(CH₂)₃CH₃ | — | 60 | 4 | | | |
| | | | B | | | | | | | |
| 2d | | 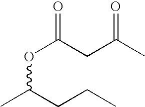 | CAL- B | O(CH₂)₂CH₃ | MTBE [50] | 30 | 4 | | | |
| 2e | | | CAL- | O(CH₂)₃CH₃ | MTBE | 30 | 4 | | | |
| | | | B | | [50] | | | | | |
| 3a | R¹ =(CH₂)₅CH₃R² = CH₃ | 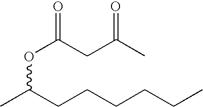 | CAL- B | OH | — | >100 | 7 | 132° C. @ 15 mbar | 178° C. @15 mbar | 46° C. |
| 3b | | | CAL- | OCH₃ | MTBE | 20 | 4 | | | |
| | | | B | | [50] | | | | | |
| 3c | | | CAL- | OCH₂CH₃ | MTBE | 20 | 5 | | | |
| | | | B | | [50] | | | | | |
| 3d | | | CAL- | O(CH₂)₂CH₃ | MTBE | 50 | 6 | | | |
| | | | B | | [50] | | | | | |
| 3e | | | CAL- | OCH(CH₃)₂ | MTBE | 60 | 8 | | | |
| | | | B | | [50] | | | | | |
| 3f | | | CAL- | O(CH₂)₃CH₃ | MTBE | >100 | 5 | | | |
| | | | B | | [50] | | | | | |
| 3g | | | CAL- | OCH₃ | — | 10 | 1 | | | |
| | | | B | | | | | | | |
| 3h | | | CAL- | OCH₂CH₃ | — | 10 | 2 | | | |
| | | | B | | | | | | | |
| 3i | | | CAL- | O(CH₂)₂CH₃ | — | 50 | 3 | | | |
| | | | B | | | | | | | |
| 3k | | | CAL- | OCH(CH₃)₂ | — | >100 | 2 | | | |
| | | | B | | | | | | | |
| 3l | | | CAL- | O(CH₂)₃CH₃ | — | >100 | 3 | | | |
| | | | B | | | | | | | |
| 4a | R¹ =CH₂(C₆H₅) R² = CH₃ | 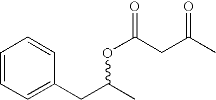 | CAL- B | OH | — | >100 | 3 | 155° C. @ 10 mbar | 109° C. @10 mbar | 46° C. |
| 4b | | | CAL- | OCH₃ | MTBE | >100 | 1 | | | |
| | | | B | | [50] | | | | | |
| 4c | | | CAL- | OCH₂CH₃ | MTBE | >100 | 1 | | | |
| | | | B | | [50] | | | | | |
| 4d | | | CAL- | O(CH₂)₂CH₃ | MTBE | >100 | 1 | | | |
| | | | B | | [50] | | | | | |
| 4e | | | CAL- | OCH(CH₃)₂ | MTBE | >100 | 2 | | | |
| | | | B | | [50] | | | | | |
| 4f | | | CAL- | O(CH₂)₃CH₃ | MTBE | >100 | 1 | | | |
| | | | B | | [50] | | | | | |
| 4g | | | CAL- | OCH₃ | — | >100 | 0.5 | | | |
| | | | B | | | | | | | |
| 4h | | | CAL- | OCH₂CH₃ | — | >100 | 1 | | | |
| | | | B | | | | | | | |
| 4i | | | CAL- | O(CH₂)₂CH₃ | — | >100 | 1 | | | |
| | | | B | | | | | | | |
| 4k | | | CAL- | OCH(CH₃)₂ | — | >100 | 1 | | | |
| | | | B | | | | | | | |
| 4l | | | CAL- | O(CH₂)₃CH₃ | — | >100 | 1 | | | |
| | | | B | | | | | | | |
| 5a | R^{1 + R}² =ortho- (CH₂)₃(C₆H₄) | 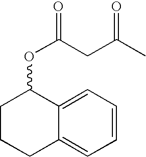 | CAL- B | OH | — | >100 | 18 | 105° C. @ 3 mbar | 104° C. @0.05 mbar | — |
| 5b | | | CAL- | OCH₃ | MTBE | >100 | 1 | | | |
| | | | B | | [50] | | | | | |
| 5c | | | CAL- | OCH₂CH₃ | MTBE | >100 | 3 | | | |
| | | | B | | [50] | | | | | |
| 5d | | 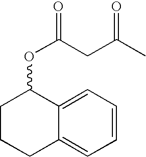 | CAL- B | O(CH₂)₂CH₃ | MTBE [50] | >100 | 2 | | | |
| 5e | | | CAL- | OCH(CH₃)₂ | MTBE | >100 | 6 | | | |
| | | | B | | [50] | | | | | |
| 5f | | | CAL- | O(CH₂)₃CH₃ | MTBE | >100 | 3 | | | |
| | | | B | | [50] | | | | | |

Chen, C.-S. et al., J. Am. Chem. Soc. 104, 7294-7299 (1982)
Legend:
CAL-B: candida antarctica lipase B
PCL: pseudomonas cepacia lipase
PFL: pseudomonas fluorescens lipase
TOF: furn over frequency
MTBE: methyl tert-butyl ether

### COMPARATIVE EXAMPLE 3

All the following examples (table (2): 6a-e) serve to illustrate the present invention further and were carried out by the general method given in example 2:

TABLE (2)

| | | | | | | | TOF |
|---|---|---|---|---|---|---|---|
| | Substituents corresponding to | | | | Cosolvent | Selectivity E by the | [mmol g⁻¹ |
| | formula (I) | Racemic substrate | Enzyme | Nu | [% (v/v)] | method of Sih¹ | h^{31 1}] |
| 6a | R¹ = C₆H₅R² = CH₃ | 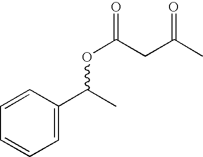 | PLE | OH | — | 2 | 32 |
| 6b | R¹ = C₆H₅R² = CH₃ | 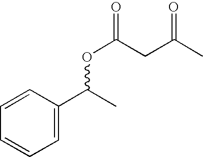 | PLE | O(CH₂)₃CH₃ | MTBE [50] | not able to be determined | 0 |
| 6c | R¹ = (CH₂)₅CH₃R² = CH₃ | 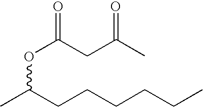 | PLE | OH | — | 2 | 200 |
| 6d | R¹ = CH₂(C₆H₅) R² = CH₃ | 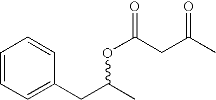 | PLE | OH | — | 2 | 54 |
| 6e | R¹ + R² =ortho-(CH₂)₃(C₆H₄) | 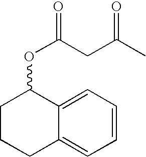 | PLE | OH | — | 2 | 63 |

Legend:
PLE: pig liver esterase

1. A process for the enantioselective preparation of secondary alcohols, comprising:
carrying out enantioselective enzymatic solvolysis of a racemic or enantiomerically enriched mixture of acetoacetic esters of chiral secondary alcohols in the presence of a nucleophile, using a lipase capable of solvolytic cleavage of an ester group.

2. The process as claimed in claim 1, wherein the racemic or enantiomerically enriched mixture comprises acetoacetic esters of the general formula (1) and (2) 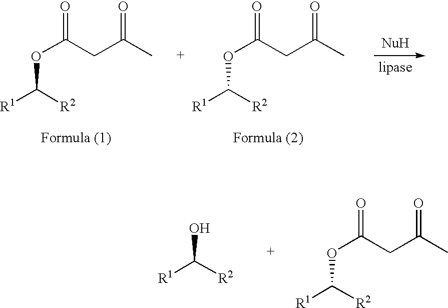
wherein R¹ is different from R² and. R¹ and R² are selected independently from the group consisting of substituted or unsubstituted. C₆-C₁₈-aryls, C₃-C₁₈-heteroaryls, C₁-C₁₈-alkyls, C₂-C₁₈-alkenyls, C₂-C₁₈-alkynyls, C₆-C₁₈-aryl-C₁-C₁₈-alkyls, C₃-C₁₈-heteroaryl-C₁-C₁₈-alkyls, C₆-C₁₈-aryl-C₁-C₈-alkenyls, C₃-C₁₈-heteroaryl-C₁-C₁₈-alkenyls, C₁-C₁₈-alkoxy-C₁-C₁₈-alkyls, C₁-C₁₈-alkoxy-C₂-C₁₈-alkenyls, C₆-C₁₈-aryloxy-C₁-C₁₈-alkyls, C₁-C₁₈-alkoxycarbonyls, hydroxycarbonyl, C₆-C₁₈-aryloxy-C₂-C₁₈-alkenyls, C₃-C₈-cycloalkyls, C₃-C₈-cycloalkyl-C₁-C₁₈-alkyls, C₃-C₈-cycloalkyl-C₂-C₁₈-alkenyls,
or R¹ and R² together with the carbon atom to which they are bound form an asymmetric, substituted, unsubstituted or heteroatom-containing cycloalkylidene,
and if the radicals R¹ and R² are substituted radicals, the substituents are selected from the group consisting of alkyl, alkenyl, alkynyl, aryl, heteroaryl, hydroxy, alkoxy, acyloxy, silyloxy, carboxylate, alkoxycarbonyl, amino, nitro and halogen radicals.

3. The process as claimed in claim 1, wherein the nucleophile has the general formula NuH, where Nu is OR⁵, SR⁵, or NR⁶R⁷ and R⁵ is selected from the group consisting of hydrogen, substituted or unsubstituted C¹-C¹⁸-alkyl, C³-C¹⁸-alkenyl, C²-C¹⁸-alkynyl, C⁶-C¹⁸-aryl-C¹-C¹⁸-alkyl, C³-C¹⁸-heteroaryl-C¹-C¹⁸-alkyl, C⁶-C¹⁸-aryl-C²-C¹⁸-alkenyl, C²-C¹⁸-heteroaryl-C²-C¹⁸-alkenyl and
wherein R¹ and R⁷ are identical or different and are selected, independently from the group consisting; of hydrogen, substituted or unsubstituted C₁-C₁₈-alkyl, C₂-C₁₈-alkenyl, C₂-C₁₈-alkynyl C₆-C₁₈-aryl, C₃-C₁₈-heteroaryl, C₆-C₁₈-aryl-C₁-C₁₈-alkyl, C₃-C₁₈-heteroaryl-C₁-C₁₈-alkyl, C₆-C₁₈-aryl-C₂-C₁₈-alkenyl, C₃-C₁₈-heteroaryl-C₂-C₁₈-alkenyl, and
if the radicals R⁵, R⁶ and R⁷ are substituted radicals, the substituents are selected from the group consisting of unsubstituted or substituted alkyl, alkenyl, alkynyl, aryl, heteroaryl, hydroxy, alkoxy, silyloxy, acyloxy, carboxylate, alkoxycarbonyl, amino, nitro and halogen radicals.

4. The process as claimed in claim 1, wherein the lipase is a Candida antarctica lipase type B (CAL-B).

5. The process as claimed in claim 1, wherein the lipase is used in free or immobilized form.

6. The process as claimed in claim 1, wherein the lipase is added in an enzyme/substrate molar ratio of from 1:1000 to 1:50,000,000.

7. The process as claimed in claim 1, wherein the nucleophile is used directly as a solvent or in mixtures with one or more other aprotic or protogenic solvents as a cosolvent.

8. The process as claimed in claim 7, wherein the nucleophile is used in mixtures with a cosolvent, and the cosolvent is selected from the group consisting of aliphatic hydrocarbons, aromatic hydrocarbons, halogenated hydrocarbons, ethers, alcohols, esters, acetonitrile and mixtures thereof.

9. The process as claimed in claim 1, wherein the nucleophile is water or a branched or unbranched C₁-C₆-alcohol.
